(19) United States
(12) Patent Application Publication
(10) Pub. No.: US 2026/0355441 A1
(43) Pub. Date: Nov. 12, 2026

(54) HEAD FOR NASAL DISPENSING OF A FLUID PRODUCT

(71) Applicant: APTAR FRANCE SAS, Le Neubourg (FR)

(72) Inventors: Youcef BELKHEROUF, Evreux (FR); Théo DUPRE, Rouen (FR); Charles CAZZOLA, Rouen (FR)

(73) Assignee: APTAR FRANCE SAS, Le Neubourg (FR)

(21) Appl. No.: 19/129,520

(22) PCT Filed: Nov. 14, 2023
(86) PCT No.: PCT/FR2023/051791

§ 371 Date: May 13, 2025

(30) Foreign Application Priority Data
Nov. 14, 2022 (FR) FR2211798
Jun. 16, 2023 (FR) FR2306234

(51) Int. Cl. A61M 15/08; A61M 11/00

(52) U.S. Cl. CPC A61M 15/08; A61M 11/007

(57) ABSTRACT

Nasal dispensing head of fluid product having a body and dispensing orifice, the body forming a reservoir containing a dose of fluid, a piston sliding in the reservoir to dispense the dose. The dispensing head has a mechanism to dispense the dose in the form of a first fluid zone inscribed in a first cylinder of diameter D1, then after a distance L from the dispensing orifice, in the form of a second fluid zone inscribed in a second cylinder of diameter D2, with D2>D1. The reservoir has a first distal cylindrical portion of length l1 and of diameter d1 and a second cylindrical portion of length l2 and of diameter d2, with d1>d2, the diameter d1 between 2 mm and 14 mm, the diameter d2 between 1 mm and 7 mm, and the ratio d2/d1 between 0.3 and 0.8, preferably of between 0.4 and 0.7.

The present invention relates to a nasal dispensing head of fluid product and a device comprising such a dispensing head.

Nasal dispensing devices are well known. They generally comprise a reservoir containing one or more doses of fluid products, and a dispensing head that is movable relative to said reservoir so as to dispense the fluid product, particularly, via a pump, a metering valve or a piston that slides in said reservoir. When the user wishes to use the device, the user inserts the dispensing head into the nostril and actuates the device so as to dispense a dose of fluid product, generally in the form of a spray.

A disadvantage with prior art devices relates to the effectiveness of the dose dispensed in the nostril, in particular, when the purpose of the dispensed fluid product is to act on the brain. In fact, only a small portion of the dose generally reaches the target zone for this type of treatment, namely the olfactory zone comprising the ethmoid sinuses, particularly, due to the shape of the spray in the nostril, which is conical at the outlet of the device. However, it appears that this conical shape is not optimal for obtaining the maximum deposition in the target zone.

Documents U.S. Pat. No. 1,155,608 and WO2018109409A1 describe nasal inhalation devices that use an external positioning device to correctly orientate the dispensing head in the user's nostril. This implementation improves the deposition of fluid product on the target zone, but this improvement remains limited due to the conical shape of the spray at the outlet of the device.

Documents US2017360831, US2011297145, WO2021069972, WO2021066195 and WO2016034803 describe other prior art devices, but none of these documents allow generating a dispensing fluid with two different fluid zones, with a second fluid zone widened relative to a first fluid zone.

The present invention aims to provide a nasal dispensing head and a device comprising such a head, which does not reproduce the above-mentioned disadvantages.

The present invention aims particularly, to provide a nasal dispensing head and a device comprising such a head, which improves the rate of deposition of active product on the olfactory zone and/or the ethmoid sinuses.

The present invention also aims to provide a nasal dispensing head and a device comprising such a head, allowing to control the shape of the spray in the nostril.

The present invention also aims to provide such a nasal dispensing head and a device comprising such a head, which are simple and inexpensive to manufacture and to assemble. The present invention therefore, provides a nasal dispensing head of fluid product comprising a body provided with a dispensing orifice, said body forming a reservoir containing a dose of fluid product, a piston sliding in a portion of said reservoir to dispense said dose of fluid product through said dispensing orifice, said dispensing head comprising means to dispense said dose of fluid product firstly in the form of a first fluid zone inscribed in a first cylinder of diameter D1, then after a distance L from said dispensing orifice, in the form of a second fluid zone inscribed in a second cylinder of diameter D2, with D2>D1, said reservoir comprising a first distal cylindrical portion of length l1 and of diameter d1 and a second proximal cylindrical portion of length l2 and of diameter d2, with d1>d2, said diameter d1 being of between 2 mm and 14 mm, preferably of between 2.9 mm and 12.5 mm, said diameter d2 being of between 1 mm and 7 mm, preferably of between 2 mm and 6 mm, and the ratio d2/d1 being of between 0.3 and 0.8, preferably of between 0.4 and 0.7.

Advantageously, said second proximal cylindrical portion extends said first distal cylindrical portion, and forms at its axial end said dispensing orifice.

Advantageously, said second proximal cylindrical portion extends said first distal cylindrical portion coaxially along a longitudinal axis of said dispensing head.

Advantageously, said piston is configured to slide in said first distal cylindrical portion between a rest position and an actuation position.

Advantageously, said piston comprises a front extension which, in the actuation position, occupies the volume of said second proximal cylindrical portion.

Advantageously, said length l1 is of between 3 mm and 20 mm, preferably of between 4 mm and 19 mm.

Advantageously, said length l2 is of between 3 mm and 18 mm, preferably of between 5 mm and 15 mm.

Advantageously, the actuation force F of said piston is of between 10N and 25N, preferably of between 12N and 22N.

Advantageously, the volume V of the dose of fluid product dispensed is of between 0.02 ml and 5 ml.

Advantageously, said diameter D1 is of between 1 mm to 3 mm, particularly about 2 mm.

Advantageously, said diameter D2 is of between 3.5 mm and 50 mm particularly of between 5 mm and 20 mm. Advantageously, the ratio D1/D2 is of between 0.05 and 0.8 particularly of between 0.1 and 0.4.

Advantageously, said first fluid zone forms an approximately axial jet through said outlet orifice, and said second fluid zone forms an approximately conical spray forming an angle α.

The present invention also provides a device for nasal dispensing of fluid product comprising a dispensing head as described above.

These features and advantages and others will become more clear in the detailed description below, made with reference to the accompanying drawings, given by way of non-limiting examples, and in which:

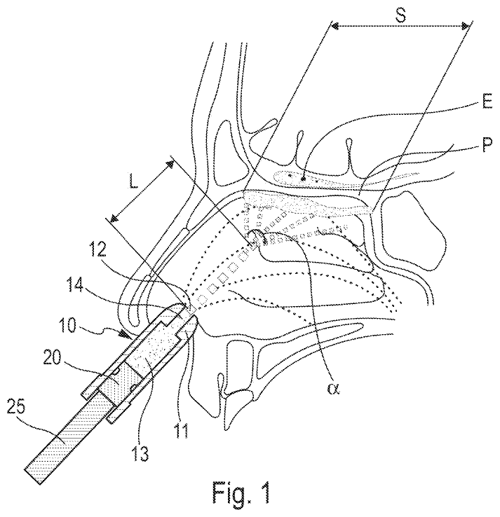

FIG. 1 is a schematic sectional view of a nasal dispensing head according a first advantageous embodiment, inserted into a nostril,

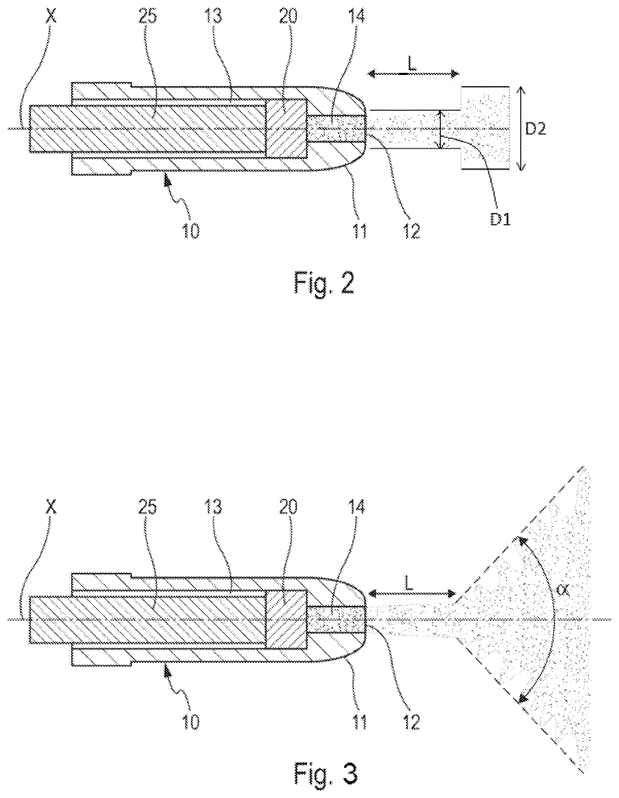

FIG. 2 is a schematic cross-sectional view of the dispensing head of FIG. 1, in the actuation position, showing the fluid zones downstream of the dispensing orifice,

FIG. 3 is a schematic cross-sectional view of the dispensing head of FIG. 2, in the actuation position, showing an advantageous spray shape downstream of the dispensing orifice,

FIG. 4 is a schematic cross-sectional view of the dispensing head of FIGS. 2 and 3, in the rest position,

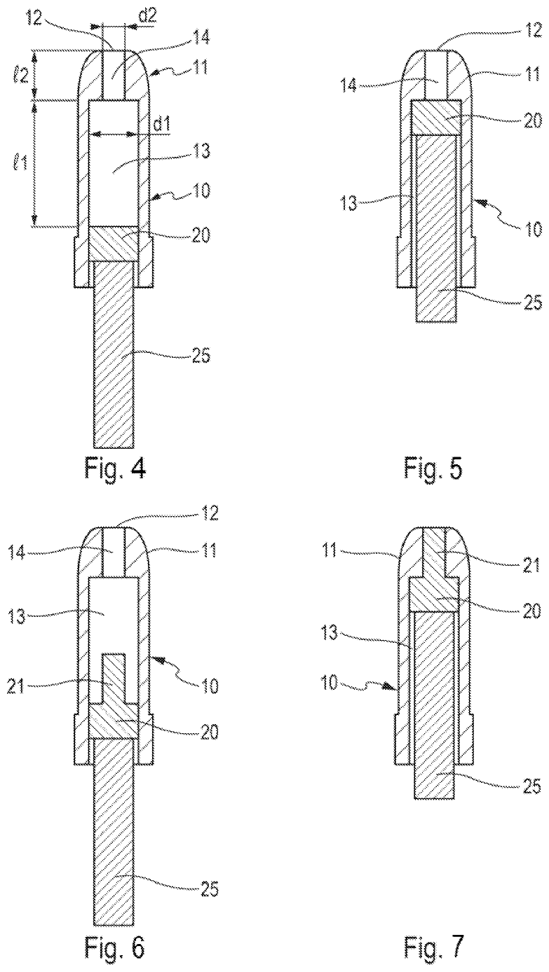

FIG. 5 is a view similar to that of FIG. 4, in the actuation position,

FIG. 6 is a schematic cross-sectional view of a nasal dispensing head according to a second, advantageous embodiment,

FIG. 7 is a view similar to that of FIG. 6, in the actuation position, and

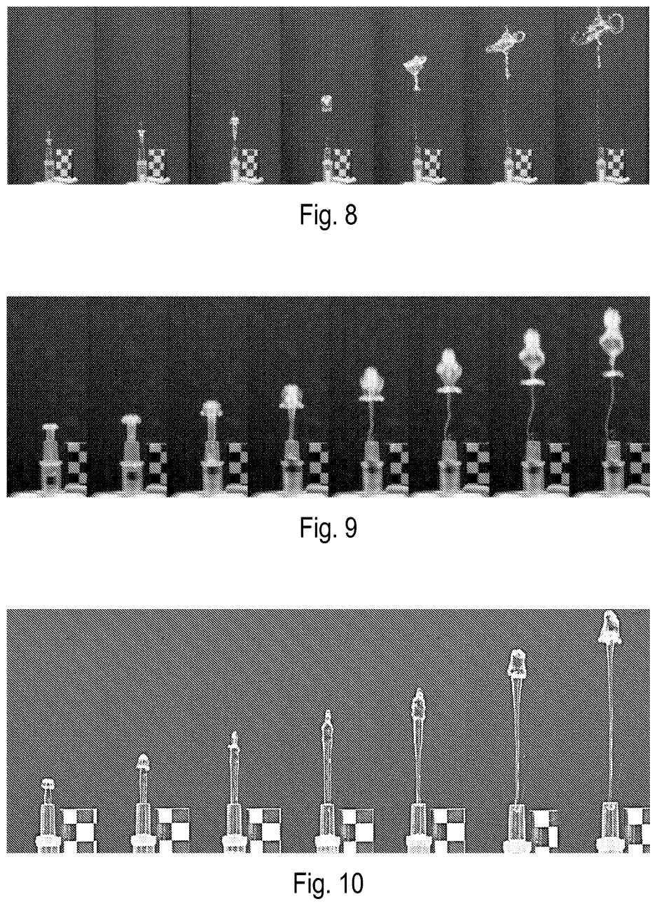

FIGS. 8 to 10 show distributions of three fluids of different viscosities.

In the description, the terms “axial” and “radial” are relative to the longitudinal axis X of the device, as shown in FIGS. 2 and 3. The terms “proximal” and “distal” refer to the dispensing orifice.

FIG. 1 shows a cross-sectional view of a nostril; The ceiling P of the nasal cavity can be seen, which comprises the ethmoid sinuses E. At the front of this ceiling P are the nasal valve and the conchae, the presence of which makes it difficult to access the ethmoid sinuses E with a conventional spray, which extends conically at the outlet of the dispensing orifice 12.

A dispensing head 10 comprising a body 11 provided with a dispensing orifice 12, is inserted into a nostril to dispense a dose of fluid product during the actuation. The dispensing head 10 contains a fluid product reservoir, with a piston 20 sliding axially in a portion of said reservoir to dispense the fluid product through the dispensing orifice.

The piston 20 is connected to an actuation rod 25 that can be actuated manually by the user.

Alternatively, the dispensing head 10 may be integrated into a dispensing device comprising dispensing means, for example, a preloaded spring, adapted to act on the actuation rod 25, particularly, on command from the user, for example, the actuation of an appropriate control button to trigger the spring. This solution has the advantage of always delivering the same actuation force, such that the properties of the spray generated during actuation are independent of the way in which the user actuates the device.

According to the invention, the fluid product is first dispensed in the form of a first fluid zone inscribed in a first cylinder of diameter D1 before transforming into a second fluid zone after a distance L from the dispensing orifice 12, said second fluid zone being inscribed in a second cylinder of diameter D2, with D2>D1.

Advantageously, as can be seen in FIG. 3, the fluid product is first dispensed in the form of an approximately axial jet before transforming into an approximately conical spray after the distance L from the dispensing orifice 12.

As can be seen in FIG. 1, the invention allows the fluid product of the first fluid zone to pass beyond the nasal valve and the conchae, for example in the form of a jet, before transforming into a second fluid zone of larger diameter, for example into a conical spray, which will allow it to reach the ethmoid sinuses E more significantly, both in quantity (part of the dose of fluid that reaches the ethmoid sinuses) and on the surface (surface S of the ethmoid sinuses affected by the fluid product).

To achieve this particular form of double fluid zones, the reservoir comprises a first distal cylindrical portion 13 of length l1 and diameter d1 and a second proximal cylindrical portion 14 of length l2 and diameter d2, with d1>d2.

The second proximal cylindrical portion 14, which extends the first distal cylindrical portion 13, forms at its axial end the dispensing orifice 12, which therefore, also has a diameter d2.

Preferably, the second proximal cylindrical portion 14 extends the first distal cylindrical portion 13 coaxially along the longitudinal axis X.

The piston 20 slides in the first distal cylindrical portion 13 between a rest position, shown in FIGS. 4 and 6, and an actuation position, shown in FIGS. 5 and 7.

In the second embodiment of FIGS. 6 and 7, the piston 20 comprises a front extension 21 which, in the actuation position, will occupy the volume of the second proximal cylindrical portion 14, thus reducing the dead volume and dispensing substantially the entire dose of fluid product contained in the reservoir 13, 14 before actuation.

Referring to the examples of FIGS. 2 and 3, at the start of actuation, a first volume of fluid product contained in the second proximal cylindrical portion 14 is expelled with a speed V1 through the dispensing orifice 12 to form a first fluid zone inscribed in a cylinder of diameter D1.

In the example of FIG. 3, this first fluid zone forms an approximately rectilinear jet.

By continuing the actuation stroke, a second volume of fluid product contained in the first distal cylindrical portion 13 will pass into the second proximal cylindrical portion 14, with the reduction of the diameter from d1 to d2 which causes an acceleration of the fluid. Thus, the second volume of fluid product from the first distal cylindrical portion 13 will be dispensed through the dispensing orifice 12 with a speed V2 greater than V1.

This second faster volume of fluid will therefore, catch up with the first slower volume of fluid and strike it at a distance L from the dispensing orifice 12, which will generate an explosion and therefore, form a second fluid zone inscribed in a cylinder of diameter D2, with D2>D1.

In the example in FIG. 3, this second fluid zone forms an approximately V-shaped spray, which defines an angle α.

Thus, the spray generated by the present invention comprises the following important characteristics for the efficiency of the device: the length L, the diameters D1 and D2, and eventually the angle α when the second fluid zone forms an approximately conical spray.

By varying the characteristics of the dispensing head 10, such as the lengths l1 and/or l2, the diameters d1 and/or d2, and/or those of the actuation of the piston, such as the actuation force of the piston 20 and/or its actuation speed, and/or the volume of the dispensed dose, it is possible to vary these parameters L, D1, D2 and a, and thus, adapt to different body types, sizes or shapes of nostrils. In particular, the length L may be chosen to generate the second wider spray zone in the right place, which necessarily varies according to the person treated, adult/child, man/woman, ethnic origins, etc. Similarly, the variation of the diameter D2 and/or of the angle α allow to modulate the surface S that will be treated, which is also variable depending on the person being treated. An adaptation of the parameters L, D1, D2 and/or a also allow, when desirable, to target an area of the nasal cavity other than the ethmoid sinuses, for example the conchae.

Another advantage of the present invention, is that it is no longer necessary to deeply insert the dispensing head 10 into the nostril, but depending on the length L, positioning the dispensing orifice 12 at the entrance of the nostril may be sufficient. Advantageously, in this case, the dispensing head 10 could comprise insertion limiting means, for example, a radial projection arranged around the body 11 of the dispensing head 10, close to the outlet orifice 12, and which could cooperate with the walls of the nostril to limit the insertion.

The dispensing head advantageously comprises the following characteristics:
the length l1 is of between 3 mm and 20 mm, preferably of between 4 mm and 19 mm;
the diameter d1 is of between 2 mm to 14 mm, preferably of between 2.9 mm to 12.5 mm;
the length l2 is of between 3 mm and 18 mm, preferably of between 5 mm and 15 mm;
the diameter d2 is of between 1 mm and 7 mm, preferably of between 2 mm and 6 mm.

Tests have shown that a significant parameter for obtaining a spray as desired is the ratio of the diameters d2/d1. Advantageously, this ratio d2/d1 is of between 0.3 to 0.8, preferably of between 0.4 to 0.7.

The combination of the dimensions d1 and d2, and the ratio d2/d1 above is required to obtain the desired effect, namely the dispensing of fluid with a double zone of fluid, with a first narrow zone and a second widened zone.

When a preloaded spring is used to move the piston and perform the actuation, the choice of the spring force has an impact on the properties of the spray generated. The actuation force the or force F of piston 20 is advantageously of between 10N and 25N, preferably of between 12N and 22N. Tests have shown that with the characteristics of the dispensing head 10 (l1, d1, l2, d2) which are constant, the greater the force F, the smaller the angle α and the greater the length L.

The volume V of the dose of fluid product dispensed is advantageously of between 0.02 ml and 5 ml.

Thus, the length L may advantageously vary between 5 mm and 50 mm, the diameter D1 may advantageously vary between 1 mm and 3 mm, the diameter D2 may advantageously vary between 3.5 mm and 50 mm, the ratio D1/D2 may advantageously vary between 0.05 and 0.8, and the angle α may advantageously vary between 15° and 125°. Advantageously, the diameter D1 is of about 2 mm, the diameter D2 may vary between 5 mm and 20 mm, and the ratio D1/D2 may vary between 0.1 and 0.4.

The table below shows different configurations tested (with water as a fluid product), to form fluid zones according to the example in FIG. 3:

| V | F | d1 | l1 | d2 | l2 | | L | |
|---|---|---|---|---|---|---|---|---|
| (ml) | (N) | (mm) | (mm) | (mm) | (mm) | d2 = d1) | (mm) | α (°) |
| 1 | 12 | 4.69 | 14 | 2 | 5 | 0.43 | 47.21 | 32.05 |
| 3 | 12 | 8.95 | 14 | 4 | 5 | 0.45 | 30.21 | 121.58 |
| 3 | 12 | 8.95 | 14 | 6 | 15 | 0.67 | 15.22 | 31.68 |
| 0.3 | 17 | 2.90 | 9 | 2 | 10 | 0.69 | 34.95 | 18.16 |
| 3 | 17 | 8.95 | 9 | 6 | 15 | 0.67 | 12.81 | 48.49 |
| 3 | 17 | 8.95 | 14 | 6 | 10 | 0.67 | 5.17 | 60.00 |
| 5 | 17 | 12.44 | 4 | 6 | 10 | 0.48 | 20.00 | 25.15 |
| 3 | 22 | 8.95 | 14 | 6 | 5 | 0.67 | 24.79 | 55.34 |

A reservoir particularly adapted to the present invention comprises the following characteristics:
volume V=0.2 ml
length l1=18.5 (±0.15) mm;
diameter d1=3.73 (±0.01) mm;
length l2=11.142 (±0.11) mm;
diameter d2=2.025 (±0.004) mm;
ratio d2/d1=0.54.

A spring particularly adapted with this reservoir, is a spring having the following characteristics:
free length=80 (±2.5) mm;
length at the start of actuation=23.5 mm;
length at the end of actuation=32.8 mm;
force F at the start of actuation (spring only)=19.8 (±0.5) N;
force F at the end of actuation (spring only)=16.5 (±0.5) N;
stiffness (spring only)=0.348 (N/mm);
force F at the start of actuation (assembled spring)=18.9 (±0.5) N;
force F at the end of actuation (assembled spring)=12.9 (±0.5) N;

This particular device was tested with three fluids of different viscosities, a first fluid (water) with a viscosity of 2 cP, a second fluid with a viscosity of 23 cP and a third fluid with a viscosity of 80 cP. These tests have demonstrated that the less viscous the fluid is, the better the spray according to the present invention.

FIG. 8 shows a spray obtained with the first fluid, FIG. 9 shows a spray obtained with the second fluid, and FIG. 10 shows a spray obtained with the third fluid.

Deposition tests in the olfactory zone were carried out with different orientations of the above device in an artificial nostril (“nasal cast”), namely with a horizontal angle AH (angle between the longitudinal axis of the device and a horizontal plane) of 30°, 45° and 60°, and with a vertical angle AV (angle between the longitudinal axis of the device and a vertical plane perpendicular to the face) of 0°, 5°, 10° and 20°. The PI insertion depth was 10 or 15 mm.

The above tests were repeated three times, and gave the following results for the deposition rates in the olfactory zone (the table contains the average of the three tests):
1. First fluid (viscosity 2 cP):

| | AH 30° | AH 45° | AH 60° |
|---|---|---|---|
| AV 0° | | PI = 15 mm: 52% | |
| AV 5° | PI = 15 mm: | PI = 10 mm: 51% | PI = 15 mm: 51% |
| | 37% | PI = 15 mm: 55% | |
| AV 10° | | PI = 15 mm: 54% | |
| AV 20° | | PI = 15 mm: 44% | |

2. Second fluid (viscosity 23 cP):

| | AH 30° | AH 45° | AH 60° |
|---|---|---|---|
| AV 5° | PI = 15 mm: | PI = 10 mm: 50% | PI = 15 mm: 56% |
| | 24% | PI = 15 mm: 57% | |

3. Third fluid (viscosity 80 cP):

| | AH 45° |
|---|---|
| AV 5º | PI = 15 mm: 57% |

By way of comparison, a standard device has been tested which generates a conical spray directly at the outlet of the device:
1. First fluid (viscosity 2 cP):

| | AH 30° | AH 45° | AH 53° |
|---|---|---|---|
| AV 5° | PI = 15 mm: 5% | PI = 15 mm: 5% | PI = 15 mm: 5% |

2. Second fluid (viscosity 23 cP):

| | AH 45° |
|---|---|
| AV 5° | PI = 15 mm: 9% |

It can be seen that the present invention dramatically increases the deposition rate at the olfactory zone, from less than 10% for a device delivering a traditional spray, to more than 50% in the majority of cases with the present invention.

A syringe on the market (Gerresheimer Gx® bulk luer cone syringe 0.5 ml) was tested for comparison. This syringe has the following characteristics:
volume V=0.5 ml
diameter d1=4.65 (±0.1) mm;
diameter d2=1.10 (±0.1) mm;
ratio d2/d1=0.24.

This syringe does not allow to obtain a dispensing of the fluid with a double zone of fluid according to the present invention.

This demonstrates the importance of the diameters d1 and d2, and especially the ratio d2/d1.

The present invention is particularly adapted for treatment of neurodegenerative diseases of the Parkinson or Alzheimer type, which require the drug to be delivered via the nose-brain barrier. Reaching the target zone, in particular, the ethmoid sinuses, via the nasal cavity is a simple and non-invasive means of administering a treatment to the brain.

The present invention may advantageously be used with the following drugs:
neurotransmitters and neuromodulators, e.g.: acetylcholine (ACH), anticholinergic drugs, adenosine triphosphate (ATP), aspartate (Asp), beta-amyloid, beta-endorphin, bradykinin, dopamine (DA), L-DOPA, Carbidopa, epinephrine, dynorphins, endomorphins, enkephalins, 5-hydroxytryptamine (5-HT), Sumatriptan, Imitrex, Migranal, Zolmitriptan, Zomig, gamma-aminobutyric acid (GABA), glutamate (glu), glycine, histamine, leptin, nerve growth factor and other growth factors, noradrenaline, nitric oxide, Substance P;
general anaesthetics, e.g.: alfentanil, desflurane, enflurane, etomidate, fentanyl, halothane, isoflurane, ketamine, methohoutletal, methoxyflurane, midazolam, lorazepam, diazepam, morphine, nitrous oxide (N2O), propofol, sevoflurane, Sufentanil, Sublimase, thiopental;
local anaesthetics, e.g.: benzocaine, bupivacaine, cocaine, lidocaine, prilocaine, procaine, ropivacaine, tetracaine;
opioid analgesics, agonist-antagonists and cough suppressants, e.g.: agonists, codeine, diphenoxylate, fentanyl, heroin and other opioids, hydrocodone, 1-alpha-acetyl-methadol, levomethadyl acetate, loperamide, meperidine, methadone, morphine, oxycodone, d-propoxyphene, combinations of opioids plus acetaminophen and asa, tramadol;
agonists/antagonists and antagonists, e.g.: buprenorphine, butorphanol, nalbuphine, nalorphine, naloxone, naltrexone, nalmefene, pentazocine, codeine, dextromethorphan, hydrocodone;
drugs used in the treatment of Parkinson's disease and motor disorders, e.g.: amantadine, apomorphine, baclofen, benzodiazepines, benztropine, bromocriptine, carbidopa, cyclobenzaprine, dantrolene, dopamine, entacapone, haloperidol, L-DOPA, pergolide, pramiprexole, ropinerole, selegiline (deprenyl), trihexyphenidyl, rasagiline, azilect, selegiline, ladostigil, rotigotine, neupro, monoamine oxidase inhibitor, COMT inhibitor;
antiepileptics, e.g. acetazolamide, carbamazepine, clonazepam, diazepam, ethosuximide, felbamate, gabapentin, lamotrigine, lorazepam, phenobarbital, phenytoin, primidone, tiagabine, topiramate, valproic acid, vigabatrin, midazolam;
drugs used in mood disorders, for example: antidepressants, amitriptyline, bupropion, citalopram, clomipramine, desipramine, fluoxetine, fluvoxamine, imipramine, nortriptyline, paroxetine, phenelzine, sertraline, trazodone, tranylcypromine, venlafaxine, antimanic drugs, carbamazepine, lithium carbonate, valproic acid;
antipsychotics (neuroleptics), e.g. chlorpromazine (CPZ), clozapine, fluphenazine, haloperidol, olanzapine, quetiapine, risperidone, sertindole, thioridazine, thiothixene, ziprasidone;
sedative-hypnotics, anxiolytics and centrally acting muscle relaxants, e.g.: alprazolam, chloral hydrate, diphenhydramine, flumazenil, flurazepam, hydroxyzine, lorazepam, oxazepam, phenobarbital, temazepam, triazolam, zaleplon, zolpidem;
drugs used in anxiety disorders and skeletal muscle relaxants, e.g.: alprazolam, chlorazepate, chlordiazepoxide, diazepam, flumazenil (antagonist), lorazepam, oxazepam;
Alzheimer's disease treatments, e.g.: donepezil, galantamine, rivastigmine, tacrine, detemir, novolin, humulin, insulin, insulin-like hormone, insulin analog such as insulin NPH, lispro, aspart, insulin detemir, glulisine, insulin glargin, insulin degludec, BDNF, GDNE, MIBG, anticancer agents, anticancer drugs, dopamine agonists, dopamine antagonists;
other drugs, e.g.: amphetamine, caffeine, ephedrine, methamphetamine, methylphenidate, phentermine, sibutramine, disulfiram, ethanol, methanol, naltrexone, atropine, scopolamine, ketamine, lysergic acid diethylamide (LSD), MDMA (methylene dioxy-methyl amphetamine), mescaline, phencyclidine (PCP), donabinol, marijuana/THC, organic solvents, nicotine, pentobarbital, neuroprotective compounds, neuroprotective peptides, neuroprotective factors, davunetide, anti-schizophrenic drugs, antidepressant drugs, comtan, entacopone, anti-TDAH agents, anti-TDAH drugs such as methylphenidate (ritalin), autism drugs and autism symptoms.

The present invention has been described with reference to advantageous embodiments, but it is understood that a person skilled in the art can provide any modifications, without going beyond the scope of the present invention, as defined by the accompanying claims.

1-15. (canceled)

16. A nasal dispensing head of fluid product comprising a body provided with a dispensing orifice, said body forming a reservoir containing a dose of fluid product, a piston sliding in a portion of said reservoir to dispense said dose of fluid product through said dispensing orifice, characterised in that said dispensing head comprises means to dispense said dose of fluid product, first in the form of a first fluid zone inscribed in a first cylinder of diameter D1, then after a distance L from said dispensing orifice, in the form of a second fluid zone inscribed in a second cylinder of diameter D2, with D2>D1, said diameter D1 being of between 1 mm and 3 mm, said diameter D2 being of between 3.5 mm and 50 mm, and the ratio D1/D2 is of between 0.05 and 0.8, said reservoir comprising a first distal cylindrical portion of length l1 and of diameter d1 and a second proximal cylindrical portion of length l2 and of diameter d2, with d1>d2, said length l1 being of between 3 mm and 20 mm, said length l2 being of between 3 mm and 18 mm, said diameter d1 being of between 2 mm and 14 mm, said diameter d2 being of between 1 mm and 7 mm, and the ratio d2/d1 being of between 0.3 and 0.8, the actuation force F of said piston being of between 10N and 25N, and the volume V of the dose of fluid product dispensed being of between 0.02 ml and 5 ml.

17. The head according to claim 16, wherein said second proximal cylindrical portion extends said first distal cylindrical portion, and forms at its axial end said dispensing orifice.

18. The head according to claim 17, wherein said second proximal cylindrical portion extends said first distal cylindrical portion coaxially along a longitudinal axis (X) of said dispensing head.

19. The head according to claim 16, wherein said piston is configured to slide in said first distal cylindrical portion between a rest position and an actuation position.

20. The head according to claim 16, wherein said piston comprises a front extension which in the actuation position occupies the volume of said second proximal cylindrical portion.

21. The head according to claim 16, wherein said length 11 is of between 4 mm and 19 mm.

22. The head according to claim 16, wherein said length l2 is of between 5 mm and 15 mm.

23. The head according to claim 16, wherein said diameter d1 is of between 2.9 mm and 12.5 mm.

24. The head according to claim 16, wherein said diameter d2 is of between 2 mm and 6 mm.

25. The head according to claim 16, wherein the ratio d2/d1 is of between 0.4 and 0.7.

26. The head according to claim 16, wherein said diameter D1 is about 2 mm.

27. The head according to claim 16, wherein said diameter D2 is of between 5 mm and 20 mm.

28. The head according to claim 16, wherein the ratio D1/D2 is of between 0.1 and 0.4.

29. The head according to claim 16, wherein said first fluid zone forms an approximately axial jet through said outlet orifice, and said second fluid zone forms an approximately conical spray forming an angle α.

30. A device for nasal dispensing of fluid product, characterised in that it comprises a dispensing head according to claim 16.
